(19) United States
(12) Patent Application Publication
(10) Pub. No.: US 2021/0282649 A1
(43) Pub. Date: Sep. 16, 2021

(54) SYSTEMS AND METHODS FOR PREDICTING HIGH FREQUENCY AND LOW FREQUENCY PATIENT PARAMETERS

(71) Applicant: Peach IntelliHealth, Inc., New York, NY (US)

(72) Inventors: L.S. Klaudyne Hong, New York, NY (US); Luigi Vacca, Weston, MA (US)

(21) Appl. No.: 17/084,419

(22) Filed: Oct. 29, 2020

Related U.S. Application Data
(60) Provisional application No. 62/929,011, filed on Oct. 31, 2019.

(51) Int. Cl. A61B 5/0205; G16H 50/20; G16H 50/30; A61B 5/00

(52) U.S. Cl. CPC A61B 5/02055; G16H 50/20; A61B 5/7267; A61B 5/742; G16H 50/30

(57) ABSTRACT

Systems and methods are disclosed for predicting values for health parameters traditionally sampled at higher frequencies (such as vital signs) and at lower frequencies (such as lab results). The higher frequency parameter predictions can take the form of a time series of predicted vital sign values and the lower frequency parameter predictions can be single predicted values based on statistical measures of past collected input features. Suitable statistical measures can include the maximum, minimum and median values.

### RELATED APPLICATIONS

The present application claims priority to and the benefit of U.S. Provisional Patent Application No. 62/929,011 entitled “SYSTEMS AND METHODS FOR PREDICTING HIGH FREQUENCY AND LOW FREQUENCY PATIENT PARAMETERS” and filed on Oct. 31, 2019, the entire contents of which are hereby incorporated by reference for all purposes.

### BACKGROUND

Vital signs (vitals) are a group of clinical signs that measures the status of a human being's vital functions. These measurements are made to assess one's physical health, and often point to the existence of one or more physical conditions when one or more of these vitals are not within the normal accepted ranges for a person with the same age, weight and gender. They can be used to assess if someone is in good health.

Temperature, heart rate, blood pressure and respiration rate are the generally accepted group of vitals. Other measurements can be added to vitals such oxygen saturation (SpO2), white blood cell count (WBC) and others for their frequency of measurement and health status assessment.

It is particularly important to be able to predict the time dynamics of vitals as they possess a great deal of information on the health status of a patient. Hospital patients have their vitals continuously monitored to make sure that in the case these degrade, patients can be admitted to intensive care unit (ICU) and proper intervention can be administered by clinical personnel. Vitals information can be combined to create new scores that measure the overall state of a patient. An example is SIRS (Systemic Inflammatory Response Syndrome), where temperature, heart rate and respiration are being used.

The measurement of body temperature is used to get an indication of core body temperature which is maintained and regulated by the body itself in order to sustain its correct functioning. Temperature changes depending on which part of the body is measured, rectal temperature is one-degree Fahrenheit higher than the oral one. It also normally varies during the day to respond to the amount of activity carried out. The average body temperature is 98.6 F (37C), but it is not uncommon to find individuals that have temperature one-degree F. lower or higher than the average.

Heart rate or pulse is the number of heart beats per minute. Its measurement is important as well the strength and dynamics of its time evolution to indicate a problem with heart function. The heart rate normally varies with age and physical conditions. It also varies during the day and night. A typical heart rate for an adult varies between 60 bpm and 100 bpm.

Blood pressure is recorded as two separate values: the systolic pressure and the diastolic or resting pressure. The systolic is higher than the diastolic. Mean arterial blood pressure is an average blood pressure taken during a single cardiac event. The normal range is between 70 and 100 mmHg. A deviation from this range can have a negative impact on the body health status.

The respiratory rate for a human body is measured by counting how many times the chest rises when a person is at rest. The typical respiration rate range is 12 to 18 bpm, except for elderly a respiration rate above 20 is not uncommon. Respiration rates may increase with medical conditions such as fever and illness.

Oxygen saturation (SpO2) is the fraction of oxygen-saturated hemoglobin relative to total hemoglobin (unsaturated+saturated) in the blood. Its normal range is 95 to 100%.

White blood cells in the blood have the task of protecting the body from infection and external substances. The normal white cell count is usually between 4×109/L and 1.1×1010/L. A high count of WBC denotes that the body is fighting disease, while a low count denotes that the immune system is weak. White blood cell count is typically collected at a lower frequency than the vital signs discussed above. In some cases it can be considered a low-frequency patient parameter.

MIMIC-III (‘Medical Information Mart for Intensive Care’) is a clinical database that contains information on ICU patients at established Boston hospitals with more than 60,000 admissions. The data was collected from hospital databases and specifically from the tables representing chart measurements, laboratory measurements, drugs, fluids, microbiology, and cumulative fluids. The patient data from the hospital databases is time-stamped and contains physiological signals and measurements, vital signs, as well as a comprehensive set of clinical data representing such quantitative data as medications taken (amounts, times, and routes), laboratory tests, measurements, and outcomes, feeding and diagnostic assessments. Each admission is characterized by a unique number called admission id. Patients are also identified by their patient ids.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example block diagram of a systems for predicting a time series of vital signs and/or future hemoglobin levels using machine learning according to some implementations.

FIG. 2 is a block diagram of an example computing system.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Vital Sign Prediction

Described herein are systems and methods for predicting future values of vital signs using predictive models. The future values of any given vital sign are predicted based on times series of values of multiple vital signs.

For example, the goal of a vitals predictive model may be to predict the vitals value of a patient in a hospital setting or outside the hospital, for example at home with a portable vitals monitor, for 12 hours where a prediction is output for every half an hour interval of that 12 hour period for a total of 24 values. In other models other total time periods with outputs of different spacings may be used without departing from the scope of this disclosure. For example, 6-24 hours' worth of vital sign predictions can be generated, with outputs for intervals ranging from 6-90 minutes in length, though the interval is preferably less than 60 minutes in length. As one specific additional non-limiting example, the model may output values for an 18 hour period using 20 minute intervals.

The use of a 12 hour space, divided into half-hour (or smaller) periods is non-standard, but it allows a more detailed picture of time evolution of the predicted vital sign. The problem of predicting such values can be satisfied by treating the data set as a statistical problem of the regression type, applied to time series. The following table shows an example time setup for a 12 hour prediction model.

| 1230am | | t = 12 | 1230pm | | | | | | 12am |
|---|---|---|---|---|---|---|---|---|---|
| 0 | 10 | (now) | 1 | 13 | 15 | 16 | 18 | 20 | 24 |

| | |
|---|---|
| inputs | Predict every half hour (discrete function) |
| every | every 30 minutes, within next 12 hours, thus |
| half | 24 predictions (each with a future time |
| hour | stamp, eg 1230pm, 1pm, 130pm . . .) |

This particular setup lends itself well to high frequency output as it reconstructs a discrete time function of the machine learning output from which a clinician or another software system can extract the maximum, minimum and other statistical measures.

In some implementations, the model uses can use 5 vital signs (heart rate, blood pressure, respiration rate, SpO₂, and body temperature) that have the power to predict themselves by sampling each vital 24 times during the arc of 12 hours. In some implementations, the model may take into account white blood cell counts sampled at the same or a lesser frequency. The predictions are made so as to follow the feature samples. So for instance, if the samples start at time t=0.5 hours and they end at time t=12, the predictions start at time 12.5 and end at time t=24. As indicated above, other time periods with other sampling intervals may also be used. For example, home health vitals monitors may output values at a lower frequency, such as once every hour, once every two hours, once every three hours, or even less frequently.

For categorical features, a value of 1 is used for positive and zero for negative. All non-categorical features were normalized to speed up the convergence of the predictive engine.

The availability of additional features in some cases leads to an enhanced prediction performance. This assumes that the added features carry dependence on the output variable. This is not always the case and when the added features do not carry such dependence, the neural net tends to overfit. For this reason, 2 approaches can be used: 1) use the Lasso regressor to trim the number of features and 2) heuristically try all combinations of features and find which combination yields the best performance. In the case of vital sign prediction, we offer an example that supports our previous statements. This example pertains to the prediction of SpO2. When we predict SpO2 using the following features: Mean BP, Temperature, Heart Rate, Respiration Rate, SpO2 and WBC with a specific machine learning model we obtain an MAE of 1.4, while if we use only Spo2 as predictive feature, we obtain an MAE of 1.1. This is due to the aforementioned problem of overfitting where one or more feature in the first case do not exhibit dependence on the output variable.

One example model uses 6 features: Temperature, Mean BP, Respiration Rate, Heart Rate, SP02, WBC and is generated using a predictive regressor model written in Python, though other programming languages could also be used. Training data was obtained from the chart table of the MIMIC3 database discussed above. Chart times were used to located the data values in time. One example of the vitals predictive model used a Multi-Layer Perceptron machine learning library called SKLEARN.NEURAL_NETWORK.MLPREGRESSOR. For the example model, every input and output node is connected to a particular time in the evolution of the model features and labels, which is believed to be unique in the health care setting.

In other implementations, other forms of regression based machine learning models may be used, including:

LinearRegression

Logistic Regression

Polynomial Regression

Stepwise Regression

Ridge Regression

Lasso Regression

ElasticNet Regression

Support Vector Regression

It is understood that other predictive algorithms or methodologies may be utilized without departing from the scope of the disclosure.

In one specific non-limiting example, the vital sign predictive model is implemented as a MLP neural net with 3 hidden layers, with 100, 50 and 10 nodes, respectively trained using stochastic gradient descent (SGD) or ADAM with adaptive learning rate. The loss objective is to minimize the mean absolute error (MAE). In alternative implementations, the loss objective is to minimize the mean squared error (MSE).

### Experimental Results

Using the above model, in a random test of 15% of the total available data, we computed the mean absolute error (MAE) for all predicted vital labels.

The total number of rows sampled was over 7 million and the training and test sizes were 85% and 15%, respectively of the total number of samples. Each row contains 24 separate records for a total of 168 million data points.

Temperature Results

The temperature MAE is 0.626. The table below shows the specific MAE for different ranges of temperatures:

TABLE 2

| Temperature Ranges | % of Data | MAE |
|---|---|---|
| Over 39.5 C. (103.1 F.) | 0.3% | 2.624 |
| Over 38 C. (100.4 F.) and below 39.5 C. (103.1 F.) | 10.4% | 1.174 |
| Between 36 C. (96.8 F.) and 38 C. (100.4 F.) | 80.3% | 0.565 |
| Below 36 C. (96.8 F.) | 8.9% | 1.072 |

Heart Rate Results

The heart rate MAE is 6.64. The table below shows the specific MAE for different ranges:

TABLE 3

| HR Ranges | % of Data | MAE |
|---|---|---|
| over 100 | 21.9% | 9.910 |
| 50 to 100 | 77.3% | 5.804 |
| below 50 | 0.8% | 11.009 |

Respiration Rate Results

The respiration rate MAE is 3.05. The table below shows the specific MAE for different ranges:

TABLE 4

| RR Ranges | % of Data | MAE |
|---|---|---|
| above 20 | 43.0% | 3.365 |
| 12 to 20 | 52.0% | 2.667 |
| below 12 | 5.0% | 5.999 |

Mean BP Results

The mean BP MAE is 7.83. The table below shows the specific MAE for different ranges:

TABLE 5

| Mean BP Ranges | % of Data | MAE |
|---|---|---|
| over 100 | 10.1% | 17.266 |
| 70 to 100 | 59.8% | 6.042 |
| below 70 | 30.1% | 8.384 |

SpO2 Results

The Sp02 MAE is 1.04 in %. The table below shows the specific MAE for different ranges:

TABLE 6

| SpO2 (in %) Ranges | % of Data | MAE |
|---|---|---|
| 95 to 100 | 82.3% | 1.134 |
| below 95 | 17.7% | 0.894 |

WBC Results

The WBC MAE is 0.849. The table below shows the specific MAE for different ranges:

TABLE 7

| WBC Ranges | % of Data | MAE |
|---|---|---|
| over 11 | 47.8% | 1.120 |
| 4.5 to 11 | 46.2% | 0.611 |
| below 4.5 | 6.0% | 0.592 |

### Conclusions

As demonstrated by the above data, a predictive model as described above (i.e., a predictive matrix comprised of 5 vitals and white blood cell count values sampled with an interval of half an hour (or less frequently for WBC) for 12 hours giving a time series of 24 data points) can successfully predict 5 vital measurements: HR, RR, Temperature, Mean BP, and SpO2, and WBC, using neural network technology. The neural net predicts a time series for 12 hours following the last sample predictive data for a total of 24 data points, each sampled half an hour apart with mean MAEs that are low compared to the typical standard deviations of the vital measurements for a wide variety of intervals. As indicated above, other neural networks using only the five above-mentioned vitals can output time series for just the five vitals.

In some implementations, an aggregate vitals score can be calculated using the output time series to provide a holistic prediction of the patients health. For example, in one implementation a vitals score or V-Score can be calculated by summing the absolute differences between predicted values for a vital at a given point in time and a measure of normal for that vital for a patient at rest. For vitals whose “normal” takes the form of the range, in various implementations, the value of normal used in the absolute value calculation may be, for example, the minimum value of the range for predicted values that fall below the normal range and the maximum value of the range for predicted values that fall above the normal range, or the middle of the normal range for all values. In some implementations, each of the vital sign differences may have a different weight attached to it. For example each vital may be weighted by the inverse of its normal standard deviation. Example weights include:
w(Heart Rate)=1/10
w(Respiration Rate)=3
w(Temperature in C)=0.4
w(Spo2)=1.5
w(MeanBp)=15

In models which output white blood cell counts, the difference between the predicted white blood cell count and the normal white blood cell count can also be included in the vital score. The white blood cell count difference can also be similarly weighted. For example, its weight may be set equal to 3.5. Each of the aforementioned weights is illustrative in nature and may change, for example, based on the demographics of the patient, or even on past historical data associated with the given patient.

In other implementations, instead of the vital score being calculated based on the sum of the weighted absolute differences, the vital score may be calculated based on the sum of changes in the absolute differences between a predicted measure of a vital sign or patient parameter and a normal value for that vital sign or patient parameter. This vital score tracks and emphasizes information about a trend in the patient's health parameters with respect to their normal levels rather than emphasizing the absolute patient health parameter values.

Hemoglobin Prediction

Clinical data measurements can be roughly divided into two classes: high-frequency and low-frequency. Examples of high-frequency measurements are body vitals: heart rate, temperature, respiration rate, spo2 and mean blood pressure. Low-frequency measurements are typically lab measurements that have frequencies of the order of 1 day or more, such as levels of bicarbonate, chloride, bun, anion, and many others. The application of machine learning predictive algorithms that require low-frequency features has to deal with 2 fundament problems: missing data and frequency variability. To remedy these problems, we propose the adoption of statistical measures and the use of data imputation methods. An example of these techniques is provided when hemoglobin is predicted using neural network technology. Such techniques, while described with respect to hemoglobin can be applied to other low frequency features, too.

Missing Data

There are 3 types of missing data: A) Missing Completely at Random (MCAR), B) Missing at Random (MAR), and C) Missing not at Random (MNAR).

MCAR data occurs when the missing data is independent of any observable or unobservable variable. MAR data occurs when the missingness looks random but in fact it is not random when all variables (observable and unobservable) are accounted for. Finally, MNAR data is neither MCAR or MAR. MNAR is the case when the value is missing due to its very value.

In clinical data measurements missing data is probably due to the fact that doctors may not deem certain clinical tests necessary given their knowledge of the patient's conditions and their expectation that the data is negative. This supports the case for MNAR. The timing of the missingness may be assigned to MCAR or MNAR. Data imputation methods can be applied to model the missing data as a function of the same feature in question in the form of point statistics (SINGLE FEATURE IMPUTATION) or as a function of all other features (MULTIVARIATE IMPUTATION).

Data Imputation Methods

Statistical Substitution

This imputation technique consists of replacing any missing value with e mean or median of that variable for all other cases. This is a simple case of univariate analysis that is simple to implement and that has the problem of not accounting for any correlations between the modelled feature and the remaining features. To carry out this type of imputation, the Python package sklearn.impute.SimpleImputer can be employed.

Iterative (Regression) Imputation

Iterative (regression) imputation is a type of multivariate imputation here each feature with missing values is modelled as a function of other features, and where the output of the regression is used for imputation. The regression is carried in an iterated round-robin fashion: at each step until the maximum number of rounds is achieved. To carry out this type of imputation, the Python package sklearn.impute.IterativeImputer can be employed.

The matrix factorization problem that we are trying to solve can be formulated as an optimization problem:

$${min}_{X , Y} {\sum }_{all observed data}^{ } {( {f}_{ut} - Xu * Yi )}^{2} + λ ( \sum {‖ Xi ‖}^{2} + {‖ Yu ‖}^{2} )$$

The feature values fᵤₜ, where the subscript u refers to an admission id and its chart time and the subscript i to a clinical feature, are being approximated by a matrix Xu*Yi, obtained by the multiplication of 2 factor matrices: Xu and Yi. The sparse matrix whose values (when present) are given by fᵤₜ has therefore dimension M×N. M is the total number of rows where each row corresponds to a given unique combination of admission id and chart time, while N is the total number of columns where each column corresponds to a specific clinical feature or group. The 2 factor matrices Xu and Yi have dimensions M×K and K×N, respectively, where K is the number of factors. Typically, the number of factors vary from 10 to 100 depending on the data, its sparseness and computation cost. To avoid overfitting a regularization term is added to the quadratic term proportionally to the constant parameter A typically less than unity.

Both the number of factors K and the parameter A can be optimized by minimizing the square loss on a test set. In practice, this may, though not necessarily, only be done for the parameter λ in the case of very large datasets and a grid of a few factor values is used.

The objective function for matrix factorization is non-convex (because of the quadratic Xu*Yi term). Stochastic gradient descent can be used to find approximate solutions to this optimization problem, however it is too slow with very large datasets. If the set of variables Xu is fixed and treated as constants, then the objective is a convex function of Yi and vice versa.

Hence, to solve the optimization problem Yi is fixed and Xu is optimized, then Xu is fixed and Yi is optimized. This is repeated until convergence. This approach is known as ALS (Alternating Least Squares). The ALS algorithm works as follows:

Initialize Xu and Yi with small random weights:

For N in Nepochs:

For u in patients:

Xu=(Σ_{fut}Yi Yi^{T}+λIₖ)⁻¹Σ_{fut}fᵤₜ

For i in Features:

Yi=(τ_{fut}Xu Xu^{T}+λIₖ)⁻¹Σ_{fut}xu fᵤₜ

Repeat until it converges below a given tolerance level.

To carry matrix factorization, the Python package called sklearn. decomposition.nmf (non-negative matrix factorization) can be used with the regularization term set to zero. For this, we have to ensure that the feature values comprising the predictive matrix are not negative, which is typically the case for clinical measurements.

A neural net predictive experiment was carried out where we have used a series of low and high frequency clinical data to predict hemoglobin with the help of data imputation.

To carry matrix factorization, the Python package called sklearn. decomposition.nmf (non-negative matrix factorization) can be used with the regularization term set to zero. For this, we have to ensure that the feature values comprising the predictive matrix are not negative, which is typically the case for clinical measurements.

A neural net predictive experiment was carried out where we have used a series of low and high frequency clinical data to predict hemoglobin with the help of data imputation.

This is the list of 29 input features selected as input features:
anion
bicarbonate
bilirubin
bun
chloride
creatinine
diastolic
gcs
glucose
heart
hematocrit
hemoglobin
Inr
Lactate
magnesium
meanbp
o2sat
ph
phosphate
platelets
potassium
pt
ptt
respiration
sodium
spo2
systolic
Temp
Wbc

These features have a wide range of frequencies and availability. The lab features will be most likely affected by the problem of missing data but even vital data may not totally exempted from it. For this reason, instead of sampling every half an hour, it is better to sample statistical measures for a certain period of time. In our case, we introduce the variable t for time in hours. In some implementations, feature values are collected for a period of 12 hours, from t=0.0 to t=12, where t=0 is the first time of the data collection for the analysis. In some implementations, fewer than 12 hours or more than 12 hours of data can be used to make a prediction. Then, in some implementations, a prediction is made for hemoglobin for a time no later than 24 after the last data point used in the prediction. In some other implementations, predictions can be made for times more than 24 hours after the time associated with the last collected data points.

The collected values of the predictive features can be used to compute the minimum value, the median and the maximum value of each feature over the data collection period. Using maximum, minimum and median values in lieu of a complete time series of data points across the same time period produces a lot fewer missing values which renders data imputation more effective. Furthermore, the median is used in lieu of the average because it is robust to outliers, which are not infrequent in healthcare data.

In the case that there is only one value present for a given feature, e.g., a lab value that is measured only a limited number of times, and in some cases only once, per day, maximum, median and minimum values are all set to that same value. If there is no value for a given feature, a data imputation method is used based on data for the patient collected prior to the given data collection period.

Output Feature: Hemoglobin

Hemoglobin is an iron protein in red-blood cells that in the human being that transport oxygen. Hemoglobin carries oxygen from the lungs to the entire body. A typical range of hemoglobin for a healthy individual is 12 to 20 grams of hemoglobin for every 100 ml of blood. A low hemoglobin count is generally defined as less than 13.5 grams of hemoglobin per deciliter of blood for men and less than 12 grams per deciliter for women. A condition of very low hemoglobin count is called anemia. High levels of hemoglobin over 17.5 grams for every ml of blood can be imputed to serious health conditions as heart failure such as. The first three most serious conditions associated with high hemoglobin count are coronary atherosclerosis, aortic valve disorder and subendocardial infarction.

Hence, the prediction of hemoglobin levels is of paramount importance as it is an indicator of the body healthiness.

The Computer Programs

For each of the three different types of imputation discussed above, a different software program is used.

With respect to the matrix factorization method, typically matrix factorization is applied to homogenous datasets with missing data. However, in this case, and in healthcare more generally, the features are often not homogeneous. For instance, maximum temperature and the minimum temperature can be considered as 2 separate and different features although they both refer to temperature. MINMAX normalization can be applied before matrix factorization imputation, leading to two beneficial results: A) negative values are removed because the NMF model works only on non-negative matrices: B) values are scaled from zero to 1 so the values are of the same order of magnitude:

Hemoglobin Model Building Using Simple Imputation Code

These are steps taken to implement a hemoglobin predictive model using simple imputation for missing data:
1) We divide the matrix feature X and the output array Y into training and test sets with a 80%, 20% split done with fixed seed.
2) We impute the missing numpy nans in the training and test matrix sets (X_train and X_test) using the Simplelmputer with strategy median.
3) We apply the package preprocessing.MinMaxScaler to scale the X_train matrix from zero to one. Separately with the same maximum and minimum imputed from X_train we apply to X_test. Min max scaling. The formula for minmax scaling is new_value=(Old Value-Min)/(Max-Min)
4) We apply the sklearn MLP (MultiLayer Perceptron) to the X_train and y_train as follows: MLPRegressor(hidden_layer_sizes=(150,50,10),
   activation=‘relu’,
   solver=‘sgd’,
   learning_rate=‘adaptive’,
   max_iter=500,
   learning_rate_init=0.00001,
   alpha=0.01,random_state=42)
5) Finally, we compute the MAE of the fitted model on the X_test.

Hemoglobin Model Building Using Matrix Factorization Imputation Code

These are steps taken to implement a hemoglobin predictive model using matrix factorization imputation for missing data:
1) We divide the matrix feature X and the output array Y into training and test sets with a 80%, 20% split done with fixed seed.
2) We apply the package preprocessing.MinMaxScaler to scale the X_train matrix from zero to one. Separately with the same maximum and minimum imputed from X_train we apply to X_test. Min max scaling. The formula for minmax scaling is new_value=(Old Value-Min)/(Max-Min) We use the MinMaxScaler first before applying the matrix factorization method because we want neighboring features to have values of the same order.
3) We model the matrix as csr sparse matrix.
4) We apply the package NMF(n_components=50,alpha=0.1, random_state=0) to both X_train and X_test separately. Possible negative values in the X_test were set to zero before the application of the package.
5) We reconstruct the approximate matrix by multiplying the 2 factor matrices W and H.
6) We apply the sklearn MLP (MultiLayer Perceptron) to the X_train and y_train as follows: MLPRegressor(hidden_layer_sizes=(150,50,10),
   activation=‘relu’,
   solver=‘sgd’,
   learning_rate=‘adaptive’,
   max_iter=500,
   learning_rate_init=0.00001,
   alpha=0.01,random_state=42)
7) Finally, we compute the MAE of the fitted model on the X_test

Hemoglobin Model Building with Iterative Imputation (Regression) Code

These are steps taken to implement a hemoglobin predictive model using iterative imputation for missing data:
1) We divide the matrix feature X and the output array Y into training and test sets with a 80%, 20% split done with fixed seed.
2) We impute the missing numpy nans in the training and test matrix sets (X_train and X_test) using the IterativeImputer with max_iter=10,n_nearest_features=3, random_state=0.
3) We apply the package preprocessing.MinMaxScaler to scale the X_train matrix from zero to one. Separately with the same maximum and minimum imputed from X_train we apply to X_test. Min max scaling. The formula for minmax scaling is new_value=(Old Value-Min)/(Max-Min)
4) We apply the sklearn MLP (MultiLayer Perceptron) to the X_train and y_train as follows: MLPRegressor(hidden_layer_sizes=(150,50,10),
   activation=‘relu’,
   solver=‘sgd’,
   learning_rate=‘adaptive’,
   max_iter=500,
   learning_rate_init=0.00001,
   alpha=0.01,random_state=42)
5) Finally, we compute the MAE of the fitted model on the X_test.

Please note that the K nearest-neighbor strategy can be also used to do data imputation, however we found the memory requirement for matrices of the size of the order and above (1M by 100)) impractical.

### Experimental Results

The best main MAE and range MAE's was obtained for the Iteration (Regression) Imputation model. These are the final results:

| | | | | | |
|---|---|---|---|---|---|
| MAE | 3.06 | hemoglobin >17.5 | | 12 | 0.014% |
| MAE | 2.71 | hemoglobin <=17.5 | | 2567 | 3.065% |
| | | and hemo >=13.5 | | | |
| MAE | 0.87 | hemoglobin <13.5 | | 81160 | 96.920% |
| Total MAE | 0.93 | | Total | 83739 | 1 |

However, the resulting MAE for matrix factorization produce results within 1% of the iterative method and therefore it should be considered a competitive method to the use of iterative regression.

### Conclusions

Three different data imputation methods along with neural net technology were employed to predict the level of hemoglobin in MIMIC patients 24 hours in advance. The best imputation method is the iterative imputation method, although the other 2 methods (median and NMF) yielded similar results.

In some implementations, the vital sign and lower-frequency health parameter (e.g., hemoglobin) predictions are made in the context of monitoring patients in an Intensive Care Unit of a hospital in order to detect potential negative health trends in advance. In some implementations, the predictions are made in the context of monitoring patients involved in clinical trials to obtain advance warning of potential adverse events. In some implementations, the predictions are used in the context of immunotherapy patient monitoring where potential for rapid decline in health would otherwise require a patient to remain in close proximity to a health care center, where the availability of predictive information may make increase a time window for such a patient to seek medical attention if there condition is predicted to worsen. In some implementations, the predictions are made in the context of monitoring patients recently released from a hospital, potentially allowing for earlier discharges given the availability of early warnings of health declines.

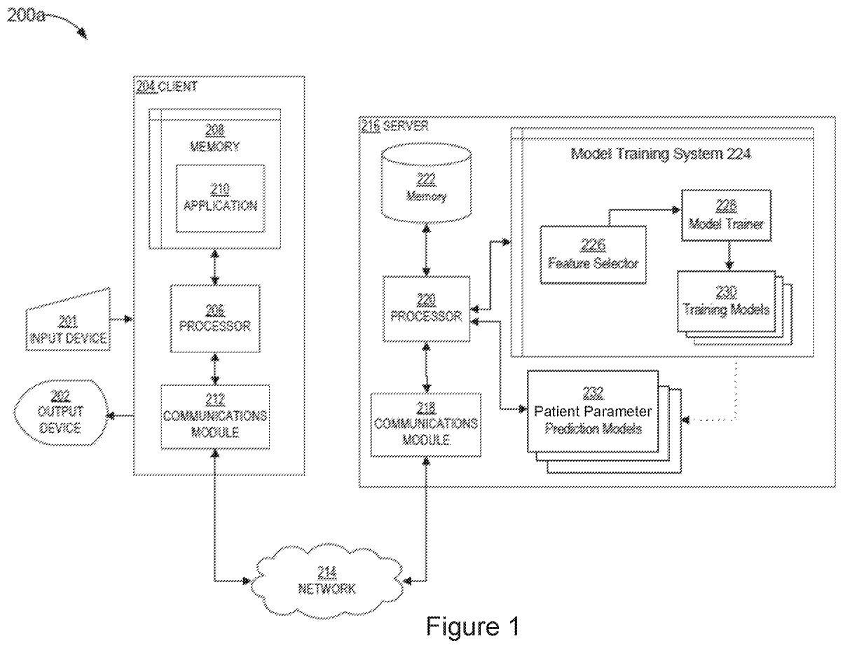

FIG. 1 is an example block diagram of a system 200a for predicting patient parameters (e.g., vitals or hemoglobin (or other lower frequency parameter) using machine learning according to some implementations. System 200a includes an input device 201 and an output device 202 coupled to a client 204. The client 204 includes a processor 206 and a memory 208 storing an application 210. The client 204 also includes a communications module 212 connected to network 214. System 200a also includes a server 216 which further includes a communications module 218, a processor 220 and a memory 222. The server 216 also includes a model training system 224. The model training system 224 includes a feature selector 226, a model trainer 228 and one or more training models 230. The server 216 also includes one or more patient parameter prediction models 232, which are shown in dotted lines to indicate that the training models 230, which were output during the training performed in the machine learning process, can be one or more patient parameter prediction models, such as the one or more patient parameter prediction models 232.

As shown in FIG. 1, the system 200a includes an input device 201. The input device 201 receives user input and provides the user input to client 204. The input device 201 may include a keyboard, mouse, microphone, stylus, and/or any other device or mechanism used to input user data or commands to an application on a client, such as client 204. In some implementations, the input device 201 may include haptic, tactile or voice recognition interfaces to receive the user input, such as on a small-format device.

The system 200a also includes a client 204. The client 204 communicates via the network 214 with the server 216. The client 204 receives input from the input device 201. The client 204 can be, for example, a large-format computing device, a small-format computing device (e.g., a smartphone or tablet), a medical data device (e.g., a small or large-format device used in a healthcare setting to collect, manage or generate patient diagnostic data or patient record data), or any other similar device having appropriate processor, memory, and communications capabilities. The client 204 may be configured to receive, transmit, and store data associated with predicting patient parameters for a patient at various amounts of time into the future.

As further shown in FIG. 1, the client 204 includes a processor 206 and a memory 208. The processor 206 operates to execute computer-readable instructions and/or data stored in memory 208 and transmit the computer-readable instructions and/or data via the communications module 212. The memory 208 may store computer-readable instructions and/or data associated with predicting a patient's parameters (e.g., vitals or low frequency parameter, such as hemoglobin level) for a specified amount of time into the future. The prediction may be a time series of values over that specified amount of time, or an individual value prediction at that period of time in the future. For example, the memory 208 may include a database of patient data, such as patient records database 115. The memory 208 includes an application 210. The application 210 may be, for example, an application to receive user input or patient data for use in determining predicted patient parameters for a given patient as discussed above. In some implementations, the application 210 may receive user input or patient data for use in determining one or more patient parameters for a given patient at a specified amount of time into the future (time series or individual value). The application 210 may include textual and graphical user interfaces to receive patient data as input and display output including predicted patient parameters for a given patient at one or more amounts of time into the future. The application 210 may include a number of configurable settings associated with triggering alerts or user notifications when one or more of the particular patient's parameters falls below or above a threshold. Additionally, or alternatively, the application 210 may output an indication, in a graphical user interface, identifying the amount of time in the future at which a parameter for a given patient is expected to exceed or fall below the applicable threshold value(s). In some implementations, the application 210 may output a list of patients for whom any predicted patient parameter is predicted to fall outside a designated safe range at one or more times in the future.

As shown in FIG. 1, the client 204 includes a communications module 212. The communications module 212 transmits the computer-readable instructions and/or patient data stored on or received by the client 204 via network 214. The network 214 connects the client 204 to the server 216. The network 214 can include, for example, any one or more of a personal area network (PAN), a local area network (LAN), a campus area network (CAN), a metropolitan area network (MAN), a wide area network (WAN), a broadband network (BBN), the Internet, and the like. Further, the network 214 can include, but is not limited to, any one or more of the following network topologies, including a bus network, a star network, a ring network, a mesh network, a star-bus network, tree or hierarchical network, and the like.

As further shown in FIG. 1, the server 216 operates to receive, store and process the computer-readable instructions and/or patient data generated and received by client 204. In some implementations, the server 216 may receive patient data directly from one or more patient monitoring devices or an electronic medical records server. The server 216 can be any device having an appropriate processor, memory, and communications capability for hosting a machine learning process. In certain aspects, the server 216 can be located on-premises with client 204, or the server 216 may be located remotely from client 204, for example in a cloud computing facility or remote data center. The server 216 includes a communications module 218 to receive the computer-readable instructions and/or patient data transmitted via network 214. The server 216 also includes one or more processors 220 configured to execute instructions that when executed cause the processors to determine predicted patient parameters for a given patient at a specified (or unspecified) amount of time into the future. The server 216 also includes a memory 222 configured to store the computer-readable instructions and/or patient data associated with predicting health parameters for a given patient at a specified (or unspecified) amount of time into the future. For example, the memory 222 may store one or more models, such as the vital sign or low-frequency health parameter prediction models 232 generated during the training of a machine learning process which have been trained to output patient parameters for patients at various amounts of time into the future. In some implementations, the memory 222 may store one or more machine learning algorithms that will be used to generate one or more training models. In some implementations, the memory 222 may store patient data that is received from client 204 and is used as a training dataset in the machine learning process in order to train a patient parameter prediction model. In some implementations, the memory 222 may store one or more trained prediction models that are used to predict vital signs or a low frequency parameter such as hemoglobin level.

As shown in FIG. 1, the server 216 includes a model training system 224. The model training system 224 functions in a machine learning process to receive patient data as training input and processes the patient data to train one or more training models. The model training system 224 includes a feature selector 226, a model trainer 228, and one or more training models 230. In some implementations, the training models 230 that are generated and output as a result of the machine learning process are configured on server 216 as standalone components on server 216. For example, the patient parameter prediction models 232 are configured on server 216 to process patient data and output a patient's parameter(s) for specified amounts of time into the future. In some implementations, the patient parameter prediction models 232 are stored in memory 222 on server 216.

The model training system 224 is configured to implement a machine learning process which will receive patient data as training input and generate a training model that can be subsequently used to predict patient parameters at specified amounts of time into the future. The components of the machine learning process operate to receive patient data as training input, select unique subsets of features within the patient data, use a machine learning algorithm to train a model based on the subset of features in the training input and generate a training model that may be output and used for future predictions based on a variety of received patient data.

As shown in FIG. 1, the model training system 224 includes a feature selector 226. The feature selector 226 operates in the machine learning process to receive patient data and select a subset of features from the patient data which will be provided as training inputs to a machine learning algorithm. In some implementations, the feature selector 226 may select a subset of features corresponding to a given patient parameter such that the machine learning algorithm will be trained to predict such parameter based on the selected subset of features. In other implementations, the feature processor 226 may select different subsets of features which do not correspond to patient data commonly used to determine parameter in question. By using a variety of training inputs, the machine learning process will generate a trained model that is able to predict a patient's parameter value from a wide variety of disparate patient data.

During the machine learning process, the feature selector 226 provides the selected subset of features to the model trainer 228 as inputs to a machine learning algorithm to generate one or more training models. A wide variety of machine learning algorithms may selected for use including algorithms such as support vector regression, ordinary least squares regression (OLSR), linear regression, logistic regression, stepwise regression, multivariate adaptive regression splines (MARS), locally estimated scatterplot smoothing (LOESS), ordinal regression, Poisson regression, fast forest quantile regression, Bayesian linear regression, neural network regression, decision forest regression, boosted decision tree regression, artificial neural networks (ANN), Bayesian statistics, case-based reasoning, Gaussian process regression, inductive logic programming, learning automata, learning vector quantization, informal fuzzy networks, conditional random fields, genetic algorithms (GA), Information Theory, support vector machine (SVM), Averaged One-Dependence Estimators (AODE), Group method of data handling (GMDH), instance-based learning, lazy learning, and Maximum Information Spanning Trees (MIST).

The model trainer 228 evaluates the machine learning algorithm's prediction performance based on patterns in the received subset of features processed as training inputs and generates one or more new training models 230. The generated training models, e.g., patient parameter prediction models 232, are then capable of receiving patient data outside of the machine learning process in which they were trained and generated to output predicted parameter values at specified amounts of time into the future for a given patient.

As further shown in FIG. 1, the patient parameter prediction models 232 that were generated as a result of performing the machine learning process, may receive patient data and process the patient data to output predicted parameter values to the processor 220. For example, the patient parameter prediction models 232, that were produced in the machine learning process, may be subsequently be included in an artificial intelligence system or application configured to receive patient data as prediction inputs and process the data to output parameter value predictions for a patient at specified amounts of time into the future. In some implementations, the processor 220 may store the predicted parameter value output from the prediction model 232 in memory 222. In some implementations, the memory 222 may store instructions to adjust or transform the received patient data based on the parameter input requirements of the prediction model. For example, the feature selector 226 may normalize values or impute missing values. In other implementations, the outputted patient parameter predictions may be forwarded to communications module 218 for transmission to the client 204 via network 214. Once received by the client 204, the outputted prediction may be transmitted to output device 202, such as a monitor, printer, portable hard drive or other storage device. In some implementations, the output device 202 may include specialized clinical diagnostic or laboratory equipment that is configured to interface with client 204 and may display the predicted parameter values in conjunction with the diagnostic or laboratory data for which the specialized clinical diagnostic or laboratory equipment is normally configured to output.

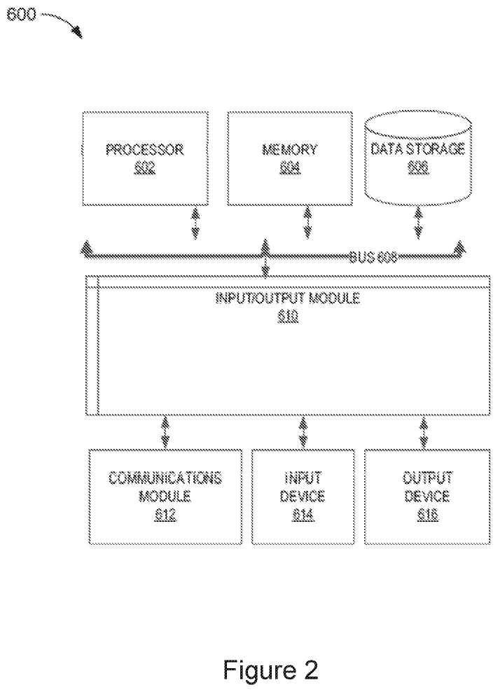

FIG. 2 is a block diagram illustrating an example computer system 600 with which the client 204, server 216, and server 202 of FIGS. 1 and 2 can be implemented. In certain aspects, the computer system 600 may be implemented using hardware or a combination of software and hardware, either in a dedicated server, or integrated into another entity, or distributed across multiple entities.

Computer system 600 (e.g., client 204, server 216, and server 202) includes a bus 608 or other communication mechanism for communicating information, and a processor 602 (e.g., processors 206 and 220) coupled with bus 608 for processing information. According to one aspect, the computer system 600 can be a cloud computing server of an IaaS that is able to support PaaS and SaaS services. According to one aspect, the computer system 600 is implemented as one or more special-purpose computing devices. The special-purpose computing device may be hard-wired to perform the disclosed techniques, or may include digital electronic devices such as one or more application-specific integrated circuits (ASICs) or field programmable gate arrays (FPGAs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, or FPGAs with custom programming to accomplish the techniques. The special-purpose computing devices may be large-format computer systems, portable computer systems, handheld devices, networking devices or any other device that incorporates hard-wired and/or program logic to implement the techniques. By way of example, the computer system 600 may be implemented with one or more processors 602. Processor 602 may be a general-purpose microprocessor, a microcontroller, a Digital Signal Processor (DSP), an ASIC, a FPGA, a Programmable Logic Device (PLD), a controller, a state machine, gated logic, discrete hardware components, or any other suitable entity that can perform calculations or other manipulations of information.

Computer system 600 can include, in addition to hardware, code that creates an execution environment for the computer program in question, e.g., code that constitutes processor firmware, a protocol stack, a database management system, an operating system, or a combination of one or more of them stored in an included memory (e.g., memory 208 or 222), such as a Random Access Memory (RAM), a flash memory, a Read Only Memory (ROM), a Programmable Read-Only Memory (PROM), an Erasable PROM (EPROM), registers, a hard disk, a removable disk, a CD-ROM, a DVD, or any other suitable storage device, coupled to bus 608 for storing information and instructions to be executed by processors 208 or 220. The processor 602 and the memory 604 can be supplemented by, or incorporated in, special purpose logic circuitry. Expansion memory may also be provided and connected to computer system 600 through input/output module 610, which may include, for example, a SIMM (Single In-Line Memory Module) card interface. Such expansion memory may provide extra storage space for computer system 600, or may also store applications or other information for computer system 600. Specifically, expansion memory may include instructions to carry out or supplement the processes described above, and may include secure information also. Thus, for example, expansion memory may be provided as a security module for computer system 600, and may be programmed with instructions that permit secure use of computer system 600. In addition, secure applications may be provided via the SIMM cards, along with additional information, such as placing identifying information on the SIMM card in a non-hackable manner.

The instructions may be stored in the memory 604 and implemented in one or more computer program products, e.g., one or more modules of computer program instructions encoded on a computer readable medium for execution by, or to control the operation of, the computer system 600 and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multi-paradigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, embeddable languages, and xml-based languages. Memory 604 may also be used for storing temporary variable or other intermediate information during execution of instructions to be executed by processor 602.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network, such as in a cloud-computing environment. The processes and logic flows described in this specification can be performed by one or more programmable processors executing one or more computer programs to perform functions by operating on input data and generating output.

Computer system 600 further includes a data storage device 606 such as a magnetic disk or optical disk, coupled to bus 608 for storing information and instructions. Computer system 600 may be coupled via input/output module 610 to various devices (e.g., device 614 or device 616. The input/output module 610 can be any input/output module. Example input/output modules 610 include data ports such as USB ports. In addition, input/output module 610 may be provided in communication with processor 602, so as to enable near area communication of computer system 600 with other devices. The input/output module 602 may provide, for example, for wired communication in some implementations, or for wireless communication in other implementations, and multiple interfaces may also be used. The input/output module 610 is configured to connect to a communications module 612. Example communications modules (e.g., communications module 612 include networking interface cards, such as Ethernet cards and modems).

The components of the system can be interconnected by any form or medium of digital data communication, e.g., a communication network. The communication network (e.g., communication network 214) can include, for example, any one or more of a personal area network (PAN), a local area network (LAN), a campus area network (CAN), a metropolitan area network (MAN), a wide area network (WAN), a broadband network (BBN), the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

For example, in certain aspects, communications module 612 can provide a two-way data communication coupling to a network link that is connected to a local network. Wireless links and wireless communication may also be implemented. Wireless communication may be provided under various modes or protocols, such as GSM (Global System for Mobile Communications), Short Message Service (SMS), Enhanced Messaging Service (EMS), or Multimedia Messaging Service (MMS), CDMA (Code Division Multiple Access), Time division multiple access (TDMA), Personal Digital Cellular (PDC), Wideband CDMA, General Packet Radio Service (GPRS), or LTE (Long-Term Evolution), among others. Such communication may occur, for example, through a radio-frequency transceiver. In addition, short-range communication may occur, such as using a BLUETOOTH, WI-FI, or other such transceiver.

In any such implementation, communications module 612 sends and receives electrical, electromagnetic or optical signals that carry digital data streams representing various types of information. The network link typically provides data communication through one or more networks to other data devices. For example, the network link of the communications module 612 may provide a connection through local network to a host computer or to data equipment operated by an Internet Service Provider (ISP). The ISP in turn provides data communication services through the world wide packet data communication network now commonly referred to as the “Internet”. The local network and Internet both use electrical, electromagnetic or optical signals that carry digital data streams. The signals through the various networks and the signals on the network link and through communications module 612, which carry the digital data to and from computer system 600, are example forms of transmission media.

Computer system 600 can send messages and receive data, including program code, through the network(s), the network link and communications module 612. In the Internet example, a server might transmit a requested code for an application program through Internet, the ISP, the local network and communications module 612. The received code may be executed by processor 602 as it is received, and/or stored in data storage 606 for later execution.

In certain aspects, the input/output module 610 is configured to connect to a plurality of devices, such as an input device 614 (e.g., input device 201) and/or an output device 616 (e.g., output device 202). Example input devices 614 include a keyboard and a pointing device, e.g., a mouse or a trackball, by which a user can provide input to the computer system 600. Other kinds of input devices 614 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Example output devices 616 include display devices, such as a LED (light emitting diode), CRT (cathode ray tube), LCD (liquid crystal display) screen, a TFT LCD (Thin-Film-Transistor Liquid Crystal Display) or an OLED (Organic Light Emitting Diode) display, for displaying information to the user. The output device 616 may comprise appropriate circuitry for driving the output device 616 to present graphical and other information to a user.

According to one aspect of the present disclosure, the client 204 and servers 216 can be implemented using a computer system 600 in response to processor 602 executing one or more sequences of one or more instructions contained in memory 604. Such instructions may be read into memory 604 from another machine-readable medium, such as data storage device 606. Execution of the sequences of instructions contained in main memory 604 causes processor 602 to perform the process steps described herein. One or more processors in a multi-processing arrangement may also be employed to execute the sequences of instructions contained in memory 604. Processor 602 may process the executable instructions and/or data structures by remotely accessing the computer program product, for example by downloading the executable instructions and/or data structures from a remote server through communications module 612 (e.g., as in a cloud-computing environment). In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. For example, some aspects of the subject matter described in this specification may be performed on a cloud-computing environment. Accordingly, in certain aspects a user of systems and methods as disclosed herein may perform at least some of the steps by accessing a cloud server through a network connection. Further, data files, circuit diagrams, performance specifications and the like resulting from the disclosure may be stored in a database server in the cloud-computing environment, or may be downloaded to a private storage device from the cloud-computing environment.

Computing system 600 can include clients and servers. A client and server are generally remote from each other and typically interact through a communication network. The relationship of client and server arises by virtue of computer programs running on the respective computers and having a client-server relationship to each other. Computer system 600 can be, for example, and without limitation, a desktop computer, laptop computer, or tablet computer. Computer system 600 can also be embedded in another device, for example, and without limitation, a mobile telephone, a personal digital assistant (PDA), a mobile audio player, a Global Positioning System (GPS) receiver, a video game console, and/or a television set top box.

The term “machine-readable storage medium” or “computer-readable medium” as used herein refers to any medium or media that participates in providing instructions or data to processor 602 for execution. The term “storage medium” as used herein refers to any non-transitory media that store data and/or instructions that cause a machine to operate in a specific fashion. Such a medium may take many forms, including, but not limited to, non-volatile media, volatile media, and transmission media. Non-volatile media include, for example, optical disks, magnetic disks, or flash memory, such as data storage device 606. Volatile media include dynamic memory, such as memory 604. Transmission media include coaxial cables, copper wire, and fiber optics, including the wires that comprise bus 608. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter affecting a machine-readable propagated signal, or a combination of one or more of them.

What is claimed is:

1. A computer-implemented method for predicting a time series of future vital signs of a patient using machine learning, the method comprising:
receiving past patient vital signs time series data, the past patient vital signs data including time series of data for a plurality of different vital signs associated with each of one or more patients;
processing the past patient vital signs data for each patient using a vital signs prediction model derived from at least one machine learning process to output for each patient a respective time series of predicted vital signs values; and
outputting on a graphical user interface, for each of the patients, the time series of predicted vital signs values.

2. The method of claim 1, wherein the past patient vital signs data time series includes data for the same number of time points as the output time series of predicted vital signs.

3. The method of claim 1, wherein values in the past patient vital signs data are separated in time by less than 60 minutes or less than 180 minutes.

4. The method of claim 3, wherein values in the past patient vital signs data are separated in time by about 30 minutes or less than 15 minutes.

5. The method of claim 1, wherein the time series for all of the received patient vital signs are used in the processing step to determine the predicted time series for each of the vital signs for each patient.

6. The method of claim 1, wherein the processing includes processing the past patient vital signs data using a trained multi-level neural network.

7. The method of claim 6, wherein the neural network is a perceptron neural network.

8. The method of claim 1, wherein the vital signs include body temperature, SpO2, respiration rate, heart rate, and blood pressure.

9. The method of claim 8, wherein the vital signs are processed along with one or more values of white blood cell count taken during the period in which the vital sign data was collected.

10. The method of claim 1, comprising calculating an aggregate vital score for each time point in the time series of predicted vital signs.

11. The method of claim 10, wherein the aggregate vital score comprises a weighted sum of absolute differences between each predicted vital sign value and a value indicative of a normal value for that vital sign.

12. The method of claim 10, wherein the aggregate vital score comprises a weighted sum of changes in absolute differences between each predicted vital sign value and a value indicative of a normal value for that vital sign.

13. The method of claim 6, further comprising training the neural network.

14. A computer-implemented method for predicting a low-frequency health parameter of a patient using machine learning, the method comprising:
receiving past values for a plurality of health parameters for the patient;
for any health parameter for which no data is collected, imputing a value for such parameter;
processing the received past values and any imputed values to identify a predicted value for a low-monitoring frequency health parameter; and
outputting on a graphical user interface, the predicted value.

15. The method of claim 14, where in the predicted value comprises a hemoglobin level.

16. The method of claim 14, wherein the predicted value comprises a hemoglobin value about 24 hours into the future from the time a last set of data points used in the processing were collected.

17. The method of claim 14, wherein processing the received past values and any imputed values comprises calculating a minimum value, a maximum value, and a median value for each health parameter and processing the minimum values, maximum values, and median values to identify the predicted value.

18. The method of claim 14, wherein the plurality of health parameters for which past values are received include health parameters sampled at a frequency of greater than once per hour and health parameters sampled at a frequency that is equal to or less than twice per day.

19. The method of claim 14, wherein imputing a value for a health parameter comprises executing an iterative regression imputation process on data collected prior to a data collection period on which the prediction is based.
